(19) United States
(12) Patent Application Publication
(10) Pub. No.: US 2026/0092928 A1
(43) Pub. Date: Apr. 2, 2026

(54) METHODS OF ANALYZING HETERODIMERIC ¿PROTEINS

(71) Applicant: Regeneron Pharmaceuticals, Inc., Tarrytown, NY (US)

(72) Inventors: Pallavi PRASAD, White Plains, NY (US); Jiann-Kae Luo, Chappaqua, NY (US); Svetlana Schussler, Montvale, NJ (US); Michelle Mazzeo, Yonkers, NY (US); Michael Perrone, Cortlandt Manor, NY (US)

(21) Appl. No.: 19/345,737

(22) Filed: Sep. 30, 2025

Related U.S. Application Data
(60) Provisional application No. 63/701,953, filed on Oct. 1, 2024.
(60) Provisional application No. 63/775,797, filed on Mar. 21, 2025.

(51) Int. Cl. G01N 33/68; B01D 15/18; B01D 15/38; B01D 15/42; B01J 20/24; B01J 20/28; B01J 20/285; B01J 20/288; G01N 30/14; G01N 30/86; G01N 30/88

(52) U.S. Cl. CPC G01N 33/6854; B01D 15/1885; B01D 15/3809; B01D 15/426; B01J 20/24; B01J 20/28004; B01J 20/28085; B01J 20/285; B01J 20/288; G01N 30/14; G01N 30/8631; G01N 30/88; G01N 2030/8831

(57) ABSTRACT

The present disclosure provides methods for analyzing a mixture of proteins using affinity chromatography. In some embodiments, the proteins are heterodimeric bispecific antibodies or antigen-binding fragments thereof. In some embodiments, guanidinium salts are used in the mobile phase of the affinity chromatography. This disclosure provides protein analysis methods that provide superior resolution and lower machine maintenance than other methods.

### CROSS-REFERENCE TO RELATED APPLICATIONS

This application claims priority to U.S. Provisional Patent Application Nos. 63/701,953, filed Oct. 1, 2024, and 63/775,797, filed Mar. 21, 2025; the entire contents of each of which is incorporated by reference herein.

### FIELD OF THE INVENTION

The present disclosure relates to methods for analyzing heterodimeric and homodimeric proteins in a mixture. Specifically, the methods relate to separating and quantitating heterodimeric proteins (including a bispecific antibody) and homodimers via affinity chromatography (including Protein A chromatography).

### BACKGROUND

The analysis and accurate quantification of protein products often requires a multi-stage process utilizing various chromatography steps to remove impurities such as host cell proteins, DNA, and undesired species of the protein product. In many cases, high concentrations of certain salts have been used to facilitate separation of a protein product from impurities. However, these salts can cause undesirable build-up and damage to separation columns and corrosion damage to chromatography equipment. This damage results in a need for frequent instrument cleaning, column replacements, and additional instrument maintenance.

Bispecific antibodies are one example of proteins for which efficient chromatography separation and isolation protocols are needed. In many instances, formation of a bispecific antibody includes combining a single common light chain and two distinct heavy chains. One of the heavy chains may contain a substituted Fc sequence (hereinafter “Fc*”) that reduces or eliminates binding of the Fc* to a protein-binding ligand such as Protein A. For example, one such Fc* sequence contains H435R/Y436F (by EU numbering system; H95R/Y96F by IMGT exon numbering system) substitutions in the CH3 domain. When producing the bispecific antibodies, co-expression of the two heavy chains and the common light chain results in three products: two of which are homodimeric for each of the heavy chains, and one of which is the desired heterodimeric bispecific product. The Fc* sequence allows selective purification of the FcFc* bispecific product on commercially available affinity columns, due to intermediate binding affinity for Protein A compared to the high avidity FcFc homodimer, or the weakly binding Fc*Fc* homodimer.

To achieve accurate quantitation of the bispecific heterodimer, good resolution between the FcFc homodimer, the Fc*Fc heterodimer, and the Fc*Fc* homodimer is required. It is also important to have methods that minimize machine maintenance costs and materials used in the analytical and purification processes, but without sacrificing resolution.

### BRIEF SUMMARY OF THE INVENTION

In one or more aspects or embodiments thereof, this disclosure provides a method of analyzing a heterodimeric protein in a mixture comprising the heterodimeric protein and impurities, the method comprising: (a) loading an affinity matrix containing a protein-binding ligand with a sample of the mixture, wherein the heterodimeric protein comprises first and second polypeptides with differing affinities for the protein-binding ligand, and wherein at least one impurity binds the protein-binding ligand with greater affinity than the heterodimeric protein and at least one impurity does not bind the protein-binding ligand or binds with lower affinity than the heterodimeric protein; (b) washing the mixture on the affinity matrix with a first mobile phase comprising a guanidinium salt at a concentration of from 50 mM to 300 mM; (c) eluting the heterodimeric protein in an eluate from the affinity matrix with a second mobile phase comprising an elution buffer; and (d) quantitating the heterodimeric protein in the eluate.

In some embodiments, the heterodimeric protein is a bispecific antibody or an antigen-binding fragment thereof. In some embodiments, the impurities comprise homodimeric proteins.

In some embodiments, the guanidinium salt is a guanidinium cation paired with an anion selected from the group consisting of SO₄²⁻, HPO₄²⁻, acetate⁻, citrate⁻, Cl⁻, NO₃⁻, ClO₃⁻, I⁻, ClO₄⁻, and SCN⁻. In some embodiments, the guanidinium salt is guanidinium chloride. In some embodiments, the concentration of the guanidinium salt is 50 to 250 mM, about 50 mM, about 100 mM, about 150 mM, about 200 mM, or about 250 mM.

In some embodiments, the first mobile phase further comprises a buffer. In some embodiments, the buffer is sodium acetate. In some embodiments, the buffer is at a concentration of from 100 mM to 300 mM. In some embodiments, the buffer is sodium acetate at a concentration of about 200 mM. In some embodiments, the first mobile phase has a pH of 8-9, or about 8.5.

In some embodiments, the elution buffer contains an acid. In some embodiments, the acid is acetic acid, or the elution buffer has a pH between 1 and 3. In some embodiments, the acetic acid is at a concentration of from 100 mM to 1 M, or the acetic acid is at a concentration of about 500 mM.

In some embodiments, the second mobile phase has a pH of from 3.5-5, 4-4.5, or 4.1-4.4.

In some embodiments, the protein-binding ligand is Protein A, and the first affinity matrix comprises a Protein A ligand affixed to a substrate. In some embodiments, the Protein A ligand is an engineered Protein A comprising a Z-domain tetramer, an engineered Protein A comprising a Y-domain tetramer, or an engineered Protein A that lacks D and E domains.

In some embodiments, the methods further include a second affinity matrix. In some embodiments, a switch valves allows mobile phase flow switching between the first affinity matrix and the second affinity matrix. In some embodiments, the second affinity matrix comprises a second protein-binding ligand, and the affinity matrix comprises a Protein G ligand affixed to a substrate Protein G.

In some embodiments, the substrate in the first or second affinity matrices comprises one or more of agarose, poly(styrene divinylbenzene), polymethacrylate, cellulose, controlled pore glass, and spherical silica.

In some embodiments, the first affinity matrix comprises a multiplicity of particles comprising a mean diameter of from 25 μm to 100 μm. In some embodiments, the second affinity matrix comprises a multiplicity of particles comprising a mean diameter of from 25 μm to 100 μm. In some embodiments, the particles comprise a mean diameter of from 40 μm to 60 μm. In some embodiments, the particles comprise a mean diameter of from 45 μm to 55 μm. In some embodiments, the particles comprise a mean diameter of about 50 μm. In some embodiments, the particles comprise pores having a mean diameter of about 1100 Å.

In some embodiments, the first polypeptide comprises a CH3 domain that is capable of binding to the protein-binding ligand and the second polypeptide comprises a CH3 domain that has reduced affinity for, or is not capable of binding to, the protein-binding ligand.

In some embodiments, the first polypeptide comprises a CH3 domain that is capable of binding to Protein A and the second polypeptide comprises a CH3 domain that is not capable of binding to Protein A. In some embodiments, the second polypeptide comprises a HY to RF substitution in its CH3 domain.

In one or more aspects or embodiments thereof, this disclosure provides a method of analyzing a heterodimeric protein in a mixture comprising the heterodimeric protein and impurities, the method comprising: (a) loading an affinity matrix containing Protein A with a sample of the mixture, wherein the heterodimeric protein comprises first and second polypeptides with differing affinities for Protein A, and wherein at least one impurity binds the Protein A with greater affinity than the heterodimeric protein and at least one impurity does not bind the Protein A or binds the Protein A with lower affinity than the heterodimeric protein; (b) washing the mixture on the affinity matrix with a first mobile phase comprising guanidinium chloride and a buffer, wherein the guanidinium chloride is at a concentration of from 50 mM to 250 mM, and wherein the buffer is sodium acetate at a concentration of from 150 mM to 250 mM; and (c) eluting the heterodimeric protein from the affinity matrix in an eluate with a second mobile phase comprising an elution buffer, wherein the elution buffer is acetic acid at a concentration of from 450 mM to 550 mM, and wherein the second mobile phase has a pH of 4.0 to 4.5; and (d) quantitating the heterodimeric protein in the eluate.

In some embodiments, the first polypeptide and the second polypeptide comprise immunoglobulin constant domains of isotype IgG1. In some embodiments, the first polypeptide and the second polypeptide comprise immunoglobulin constant domains of isotype IgG4.

In some embodiments, the heterodimeric protein is a bispecific antibody, wherein the bispecific antibody is an immunoglobulin molecule comprising four polypeptide chains, wherein the four polypeptide chains include two heavy chains and two light chains inter-connected by disulfide bonds, wherein each heavy chain comprises a heavy chain variable region and a heavy chain constant region comprising CH1, CH2 and CH3 domains, and wherein each light chain comprises a light chain variable region and a light chain constant region.

In one or more aspects or embodiments thereof, this disclosure provides a method of analyzing a heterodimeric protein and two types of homodimeric proteins in a mixture comprising the heterodimeric protein and the two types of homodimeric proteins, the method comprising: (a) loading a first affinity matrix containing a first protein-binding ligand with a sample of the mixture, wherein the heterodimeric protein comprises first and second polypeptides with differing affinities for the first protein-binding ligand, wherein a first type of homodimeric protein binds the protein-binding ligand with greater affinity than the heterodimeric protein and a second type of homodimeric protein does not bind the protein-binding ligand or binds with lower affinity than the heterodimeric protein, and wherein the first protein-binding ligand is Protein A; (b) loading a second affinity matrix containing a second protein-binding ligand with a sample of the mixture, wherein the second type of homodimeric protein binds the second protein-binding ligand with greater affinity than the heterodimeric protein, and wherein the second protein-binding ligand is Protein G; (c) washing the mixture on the affinity matrix with a first mobile phase comprising a guanidinium salt at a concentration of from 50 mM to 300 mM and a buffer, wherein the buffer is sodium acetate at a concentration of from 100 to 300 mM sodium acetate; (d) eluting the heterodimeric protein and the first type of homodimeric protein from the first affinity matrix with a second mobile phase comprising an elution buffer, wherein the elution buffer comprises acetic acid at a concentration of from 300 mM to 700 mM and the elution buffer is mixed with the first mobile phase in increasing volumes to decrease pH of the second mobile phase over time; (e) eluting the second type of homodimeric protein from the second affinity matrix; and (f) quantitating the first type of homodimeric protein, the heterodimeric protein, and the second type of homodimeric protein.

In some embodiments, switch valves are used to switch flow of the mobile phases through the first affinity matrix and the second affinity matrix during the elution process. In some embodiments, the switch valve is used to allow flow through the first affinity matrix for the elution of the heterodimeric protein and the first type of homodimeric protein, and the switch value is then used to allow flow through the second affinity matrix for the elution of the second type of the homodimeric protein.

In some embodiments, the heterodimeric protein is a bispecific antibody or antigen-binding fragment thereof. In some embodiments, the first type of homodimeric protein is a FcFc antibody. In some embodiments, the second type of homodimeric protein is a Fc*Fc* antibody.

In some embodiments, quantitating the first type of homodimeric protein, the heterodimeric protein, and the second type of homodimeric protein comprises measuring the area under the curve of peaks corresponding to the proteins in a chromatogram.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

### BRIEF DESCRIPTION OF THE DRAWINGS

FIG. 1 is a schematic showing the structural differences between a homodimeric antibody impurity Fc*Fc* and a homodimeric antibody impurity FcFc compared to a desired heterodimeric bispecific antibody Fc*Fc.

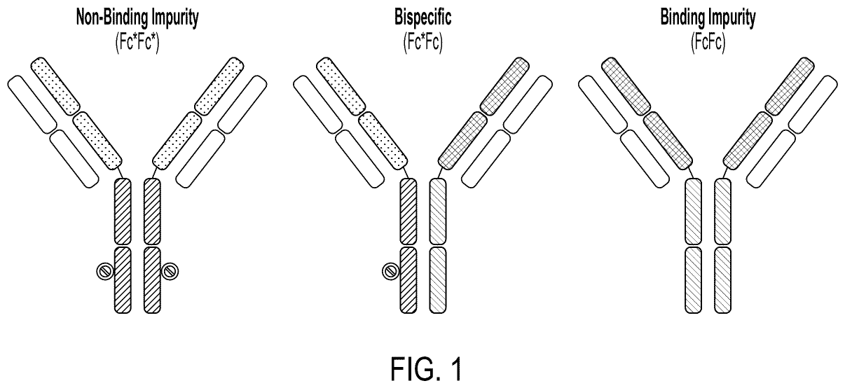

FIG. 2 shows a chromatogram overlay demonstrating comparable peak resolution and separation between methods using GdmCl in the mobile phase compared to methods using higher concentrations of other salts in the mobile phase, where the details for Method 1 and New Method 1 are summarized in Table 1.

FIG. 3 shows chromatogram overlays of the analysis of a bispecific antibody revealing that GdmCl is an effective chaotropic substitute for other salts.

FIG. 4 shows that the presently disclosed methods produce consistent separation and peak resolution results over multiple injections, confirming that the methods disclosed herein require fewer column replacements and less machine maintenance.

FIG. 5 shows the screening of various GdmCl concentrations used in the mobile phase and their respective impact on the resolution and peak area of the target bispecific antibody.

FIG. 6 shows a comparison of antibody species quantitation between methods using CaCl₂) or GdmCl as the chaotropic agent in the mobile phase and demonstrates comparable quantitation of the three species between the analytical methods.

FIG. 7 shows percent peak area between the three species of antibodies using methods with GdmCl as the chaotropic agent based on data from over 500 injections over a period of 25 days.

### DETAILED DESCRIPTION

Before the present invention is described, it is to be understood that this disclosure is not limited to particular methods and experimental conditions described, as such methods and conditions may vary. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### General

The present invention is predicated, at least in part, on the discovery that guanidinium salts provide superior value as a mobile phase modifier as compared to other chaotropic salts, such as NaCl, CaCl₂) or MgCl₂. The analytical methods provided herein, with guanidinium salt as a mobile phase modifier in combination with other mobile phase components, reduce machine maintenance costs and the footprint of the analytical system for large-scale commercial manufacturing without sacrificing target peak separation and resolution. Cost of materials and efficient analysis of therapeutic bispecific antibodies are significant concerns in the production of bispecific antibodies. For example, the cost of replacing a 100 L separation column can easily exceed $1.5 million USD, and delay manufacturing processes. Thus, extending the usable lifetime of an affinity chromatography column over a greater number of cycles can achieve significant cost advantages. Being able to maintain uncompromised chromatography columns for longer periods of use both minimizes the cost of the column materials (e.g., chromatography medium or resin) as well as the space occupied by the equipment necessary to achieve a desired product. Separation of bispecific antibodies via affinity chromatography has been described previously, but these processes generally make use of salts (e.g., sodium chloride and/or calcium chloride) in the mobile phase that can quickly damage columns. The present inventors have discovered that by replacing these salts with a guanidinium salt, overall maintenance costs can be significantly reduced, while still achieving quality separation and analysis of the heterodimer and the homodimeric impurities. Overall, the methods described herein provide effective separation and analysis of target compounds and non-target compounds without damaging expensive chromatography equipment.

### Definitions

The term “antibody”, as used herein, includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹ M; at least 10⁻¹¹ M; or at least 10⁻¹² M, as measured by surface plasmon resonance, e.g., BIACORE™ or solution-affinity ELISA.

The term “analyzing”, as used herein, can include separating, isolating, characterizing, and/or measuring quantities or qualitative traits of proteins or compounds of interest. For a non-limiting example, “analyzing” includes quantitating the respective amounts of FcFc, FcFc*, and Fc*Fc* proteins in a given sample.

The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

In various embodiments of the methods discussed herein, the heterodimeric proteins, bispecific antibodies, Fc-containing proteins, or the like, may be of isotype IgG. In some cases, the heterodimeric proteins, bispecific antibodies, Fc-containing proteins, or the like, are of isotype IgG1, IgG2, IgG3 or IgG4. In some cases, the heterodimeric proteins, bispecific antibodies, Fc-containing proteins, or the like are of isotype IgG1. In some cases, the heterodimeric proteins, bispecific antibodies, Fc-containing proteins, or the like, are of isotype IgG4. In various embodiments, the heterodimeric proteins, bispecific antibodies, Fc-containing proteins, or the like, are fully human.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with this invention include those, e.g., that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The phrase “Fc-containing protein” includes antibodies, bispecific antibodies, heterodimeric proteins and immunoadhesins, and other binding proteins that comprise at least a functional portion of an immunoglobulin CH2 and CH3 region. A “functional portion” refers to a CH2 and CH3 region that can bind a Fc receptor (e.g., an FcγR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcγR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 2591 (e.g., V2591), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “star substitution”, “Fc*”, and “HC*” includes any molecule, immunoglobulin heavy chain, Fc fragment, Fc-containing molecule, heterodimeric protein and the like which contain a sequence within the CH3 domain that reduces or abrogates binding to Protein A. Specific modifications, such as H95R and Y96F, that can diminish or abrogate Protein A binding in the CH3 domain are discussed in U.S. Pat. No. 8,586,713. This dipeptide mutation is designated as the “star substitution”.

The term “cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g. a retinal cell that expresses a viral gene (e.g., a PER.C6™ cell).

The phrase “mobile phase modifier” includes moieties that reduce the effect of, or disrupt, non-specific (i.e., non-affinity) ionic and other non-covalent interactions between proteins. “Mobile phase modifiers” can include, for example, salts, ionic combinations of Group I and Group II metals with acetate, bicarbonate, carbonate, a halogen (e.g., chloride or fluoride), nitrate, phosphate, or sulfate. A non-limiting illustrative list of “mobile phase modifiers” includes beryllium, lithium, sodium, and potassium salts of acetate; sodium and potassium bicarbonates; lithium, sodium, potassium, and cesium carbonates; lithium, sodium, potassium, cesium, and magnesium chlorides; sodium and potassium fluorides; sodium, potassium, and calcium nitrates; sodium and potassium phosphates; and calcium and magnesium sulfates.

“Mobile phase modifiers” also include chaotropic agents, which weaken or otherwise interfere with non-covalent forces and increase entropy within biomolecular systems. Non-limiting examples of chaotropic agents include butanol, calcium chloride, ethanol, guanidinium chloride, lithium perchlorate, lithium acetate, magnesium chloride, phenol, propanol, sodium dodecyl sulfate, thiourea, and urea. Chaotropic agents include salts that affect the solubility of proteins.

“Mobile phase modifiers” include those moieties that affect ionic or other non-covalent interactions that, upon addition to a pH gradient or step, or upon equilibration of a Protein A support in a “mobile phase modifier” and application of a pH step or gradient, results in a broadening of pH unit distance between elution of a homodimeric IgG and a heterodimeric IgG (e.g., a wild-type human IgG and the same IgG but bearing one or more modifications of its CH3 domain as described herein). A suitable concentration of a “mobile phase modifier” can be determined by its concentration employing the same column, pH step or gradient, with increasing concentration of “mobile phase modifier” until a maximal pH distance is reached at a given pH step or pH gradient. “Mobile phase modifiers” may also include non-polar modifiers, including for example propylene glycol, ethylene glycol, and the like.

As used herein, “affinity chromatography” is a chromatographic method that makes use of the specific, reversible interactions between biomolecules rather than general properties of the biomolecule such as isoelectric point, hydrophobicity, or size, to effect chromatographic separation. “Protein A affinity chromatography” or “Protein A chromatography” refers to a specific affinity chromatographic method that makes use of the affinity of the IgG binding domains of Protein A for the Fc portion of an immunoglobulin molecule. This Fc portion comprises human or animal immunoglobulin constant domains CH2 and CH3 or immunoglobulin domains substantially similar to these. Protein A encompasses native protein from the cell wall of Staphylococcus aureus, Protein A produced by recombinant or synthetic methods, and variants that retain the ability to bind to an Fc region. In practice, Protein A chromatography involves using Protein A immobilized to a solid support. See Gagnon, Protein A Affinity Chromatography, Purification Tools for Monoclonal Antibodies, pp. 155-198, Validated Biosystems, 1996. Protein G and Protein L may also be used for affinity chromatography. The solid support is a non-aqueous matrix onto which Protein A adheres. Such supports include agarose, sepharose, glass, silica, polystyrene, nitrocellulose, charcoal, sand, cellulose and any other suitable material. Such materials are well known in the art. Any suitable method can be used to affix the second protein to the solid support. Methods for affixing proteins to suitable solid supports are well known in the art. See e.g. Ostrove, in Guide to Protein Purification, Methods in Enzymology, 182:357-371, 1990. Such solid supports, with and without immobilized Protein A, are readily available from many commercial sources including such as Vector Laboratory (Burlingame, Calif.), Santa Cruz Biotechnology (Santa Cruz, Calif.), BioRad (Hercules, Calif.), Amersham Biosciences (part of GE Healthcare, Uppsala, Sweden), Pall (Port Washington, NY) and EMD-Millipore (Billerica, Mass.). Protein A immobilized to a pore glass matrix is commercially available as PROSEP®-A (Millipore). The solid phase may also be an agarose-based matrix. Protein A immobilized on an agarose matrix is commercially available as MABSELECT™ (Cytiva) or MABSELECT SURE™ (Cytiva) or MABSELECT SURE PCC™ (Cytiva).

Affinity chromatography also includes media that can be used to selectively bind and thus purify or quantitate antibodies, fragments of antibodies, or chimeric fusion proteins that contain immunoglobulin domains and/or sequences. Antibodies include IgG, IgA, IgM, IgY, IgD and IgE types. Antibodies also include single chain antibodies such as camelid antibodies, engineered camelid antibodies, single chain antibodies, single-domain antibodies, nanobodies, and the like. Antibody fragments include VH, VL, CL, CH sequences. Antibody fragments and fusion proteins containing antibody sequences include for example F(ab′)₃, F(ab′)₂, Fab, Fc, Fv, dsFv, (scFv) 2, scFv, scAb, minibody, diabody, triabody, tetrabody, Fc-fusion proteins, trap molecules, and the like (see Ayyar et al., Methods 56 (2012): 116-129). Such affinity chromatography media may contain ligands that selectively bind antibodies, their fragments, and fusion proteins contains those fragments. Such ligands include antibody binding proteins, bacterially derived receptors, antigens, lectins or anti-antibodies directed to the target molecule. the antibody requiring purification. For example, camelid-derived affinity ligands directed against any one or more of IgG-CH1, IgG-Fc, IgG-CH3, IgG1, LC-kappa, LC-lambda, IgG3/4, IgA, IgM, and the like may be used as affinity ligands (commercially available as CAPTURESELECT chromatography resins, Life Technologies, Inc., Carlsbad, Calif.)

### Guanidinium Salts

Guanidinium salts include the salts of guanidine. Guanidine is a nitrogen containing organic compound found naturally in many plants and animals. To form a salt, the guanidinium cation can be paired with different anions. For example, a guanidinium cation can be paired with SO₄²⁻, HPO₄²⁻, acetate⁻, citrate⁻, Cl⁻, NO₃⁻, ClO₃⁻, I⁻, ClO₄⁻, or SCN⁻ to form a salt. In some embodiments of the methods disclosed herein, the guanidinium salt used is guanidinium chloride (GdmCl). Different salts have different characteristics of surface tension, solubility, ability to denature proteins, and ability to stabilize proteins. These characteristics have been organized and can be assessed by a person of skill in the art using the Hofmeister series classifications.

### Methods of Analyzing a Mixture of Proteins

Embodiments of methods of the present invention include the steps discussed in Example 1. The methods of analyzing a mixture of proteins include (a) loading a mixture of heterodimeric proteins, homodimeric proteins, and impurities onto an affinity matrix; (b) washing the affinity matrix with a wash buffer containing GdmCl; and (c) measuring the amounts of the heterodimeric and homodimeric proteins. In some embodiments, the methods further comprise an initial equilibration step. In some embodiments, the methods further comprises separating and eluting the heterodimeric and/or homodimeric proteins. In some embodiments, the amounts of the heterodimeric and homodimeric proteins are measured via the area-under-the-curve of the corresponding peaks in a chromatogram produced during the chromatography analysis. As used herein, the term “analyzing” can mean separating, measuring, characterizing, and/or isolating proteins of interest.

In various embodiments, loading the mixture of heterodimeric protein and impurities onto the affinity matrix includes loading clarified cell culture from one or more bioreactors containing the cells expressing the nucleotide sequences encoding the heterodimeric protein. For example, the cells may express the nucleotides encoding each of the heavy and light chains forming a bispecific antibody (e.g., a CD3×CD20 bispecific antibody, a MET×MET bispecific antibody in which the two arms bind distinct epitopes of MET, a CD3×BCMA bispecific antibody, a CD22×CD28 bispecific antibody, a PSMA×CD28 bispecific antibody, a CD3×PSMA bispecific antibody, a CD3×MUC16 bispecific antibody, a CD3×STEAP2 bispecific antibody, or the like). In some cases, each of the antigen-binding arms of the bispecific antibody comprises a common light chain. The clarified cell culture will include the heterodimeric protein (e.g., bispecific antibody), along with the homodimeric protein species, other host cell proteins, and nucleic acids such as RNA and DNA. In some cases, the heterodimeric protein may be produced in eukaryotic cells, such as for example Chinese hamster ovary (CHO) cells.

In some embodiments, the mixture loaded onto the affinity matrix includes a mixture of proteins containing (i) a first homodimer comprising two copies of a first polypeptide, (ii) a heterodimer comprising the first polypeptide and a second polypeptide, and (iii) a second homodimer comprising two copies of the second polypeptide. The first and second polypeptides have different affinities for the affinity matrix, such that the first homodimer, the heterodimer and the second homodimer can be separated on the basis of differential binding to the affinity matrix. Differential binding to an affinity matrix can be manipulated by changing, inter alia, the pH and/or ionic strength of a solution passed over the affinity matrix.

Following loading of the clarified cell culture, the affinity matrix is washed with a wash buffer comprising GdmCl (e.g., at a concentration of from about 50 mM to about 250 mM) and a pH of from 5 to 9. In some cases, the pH of the wash buffer is from 6 to 9. In some cases, the pH of the wash buffer is from about 8 to about 9. In various embodiments, the pH of the wash buffer is or is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In some embodiments, the pH of the wash buffer is or is about 8.5. In various embodiments, the buffer can be any buffer capable of maintaining the pH at the desired point or within the desired range. In various embodiments, the buffer concentration may be from about 10 mM to about 300 mM. In some cases, the buffer concentration is from about 100 mM to about 250 mM. In some cases, the buffer concentration is from about 150 mM to about 225 mM. In some cases, the buffer concentration is from about 175 mM to about 210 mM. In some cases, the buffer concentration is from about 200 mM. In various embodiments, the buffer concentration is or is about 5 mM, 10 mM, 25 mM, 50 mM, 75 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM. In some embodiments, the wash buffer concentration is or is about 200 mM. In some embodiments, the wash buffer is sodium acetate.

In some cases, the wash buffer comprises salt at a concentration of from about 50 mM to about 300 mM. In some cases, the wash buffer comprises salt at a concentration of from about 50 mM to about 250 mM. In some cases, the wash buffer comprises salt at a concentration of from about 50 mM to less than 250 mM. In some cases, the wash buffer comprises salt at a concentration of, or of about, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 241 mM, 242 mM, 243 mM, 244 mM, 245 mM, 246 mM, 247 mM, 248 mM, 249 mM, 250 mM, 251 mM, 252 mM, 253 mM, 254 mM, 255 mM, 256 mM, 257 mM, 258 mM, 259 mM, 260 mM, 270 mM, 280 mM, 290 mM, or 300 mM. In some embodiments, the salt concentration of the wash buffer is, or is about, 250 mM. In some embodiments, the wash buffer comprises about 250 mM GdmCl.

Following the wash or washes discussed above, the heterodimeric protein is eluted from the affinity matrix with an elution buffer or strip buffer. The elution buffer has a pH of from about 2 to 4, and includes an acid (e.g., at a concentration of greater than 200 mM). In some embodiments, the pH of the elution buffer is from about 2.1 to about 3.5. In some embodiments, the pH of the elution buffer is from about 2.2 to about 3. In various embodiments, the pH of the elution buffer is or is about 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4. In some embodiments, the pH of the elution buffer is 2.0. In some embodiments, the pH of the elution buffer is 2.4. In various embodiments, the acid used in the elution buffer can be any acid capable of maintaining the pH at the desired point or within the desired range. In various embodiments, the acid concentration may be from about 50 mM to about 800 mM. In some cases, the acid concentration is from about 100 mM to about 700 mM. In some cases, the acid concentration is from about 200 mM to about 600 mM. In various embodiments, the acid concentration is or is about 100 mM, 150 mM, 200 mM, 250 mM, 300 mM, 350 mM, 400 mM, 410 mM, 420 mM, 430 mM, 440 mM, 450 mM, 460 mM, 470 mM, 480 mM, 490 mM, 495 mM, 500 mM, 505 mm, 510 mM, 520 mM, 530 mM, 540 mM, 550 mM, 560 mM, 570 mM, 580 mM, 590 mM, 600 mM, 625 mM, 650 mM, 675 mM, 700 mM, 750 mM, 800 mM, 850 mM, 900 mM, 950 mM or 1000 mM (1 M). In some embodiments, the acid concentration in the elution buffer is or is about 500 mM. In some embodiments, the acid is acetic acid.

The elution buffer can mixed with the first mobile phase, thereby generating a second mobile phase capable of promoting elution. Alternatively, a second mobile phase comprising elution buffer can be used to elute proteins from the affinity matrix. In some embodiments, the second mobile phase comprising elution buffer has a pH of 2 to 5. In some embodiments, the second mobile phase has a pH of from 3.5 to 5. In some embodiments, the second mobile phase has a pH of from 4 to 4.5. In some embodiments, the second mobile phase has a pH of from 4.1 to 4.4. In some embodiments, the second mobile phase has a pH of about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5. In some embodiments, the second mobile increases in acidity over time as more elution buffer is mixed with the first mobile phase during the chromatography run.

After elution of the target and non-target molecules from the affinity matrix using the elution buffer and corresponding mobile phase, the affinity matrix can re-equilibrated to a pH of from 5 to 9. In various embodiments, the affinity matrix is re-equilibrated to a pH of or of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In some embodiments, the affinity matrix is re-equilibrated to a pH of about 8.5. Equilibration can be performed with an equilibration buffer having the desired pH. In various embodiments, the buffer can be any buffer capable of maintaining the pH at the desired point or within the desired range.

In various embodiments, loading of the affinity matrix from clarified cell culture or from the neutralized eluate containing the heterodimeric protein can include addition of material of up to about 75 g/L of affinity matrix resin. In various embodiments, the affinity matrix is loaded with less than or equal to 65 g/L, 60 g/L, 55 g/L or 50 g/L of material.

In some embodiments, the affinity matrix comprises a ligand (e.g., Protein A or Protein G) affixed to a substrate. In some cases, the substrate is a bead or particle, such that the affinity matrix is a plurality of particles affixed with the ligand. In various embodiments, the ligand is Protein A or Protein G, which are both examples of IgG binders. When the ligand is Protein A, the Protein A may be a naturally occurring or modified Staphylococcal Protein A, or it may be an engineered Protein A. Engineered Protein A may be for example a Z-domain tetramer, a Y-domain tetramer, or an engineered Protein A that lacks D and E domains. These engineered Protein A exemplars are unable to bind (or bind with very low affinity if at all) to the VH3 domain of an immunoglobulin, but can still bind to the CH3 domains of IgG1, IgG2 and IgG4.

In some cases, the affinity matrix substrate contains or is made of agarose, poly(styrene divinylbenzene), polymethacrylate, controlled pore glass, spherical silica, cellulose and the like. In the embodiments in which the substrate is shaped as a bead or particle, the mean diameter of the particles is from 25 μm to 100 μm. In some embodiments, the mean diameter of the particles is from about 40 μm to about 60 μm. In some embodiments, the mean diameter of the particles is from about 45 μm to about 55 μm. In some embodiments, the mean diameter of the particles is from about 40, 41, 42, 43, 44, 45, 46, 47, 48, 49, 50, 51, 52, 53, 54 or 55 μm. In some cases, the mean diameter of the particles is about 45 μm. In some cases, the mean diameter of the particles is about 50 μm. In some embodiments, the particles have a mean diameter of 35 μm, 45 μm, 60 μm, 75 μm, or 85 μm. In some embodiments, the particles contain pores having a mean diameter of about 1000 Å, 1050 Å, 1100 Å, 1150 Å or 1200 Å. In some embodiments, the particles contain pores having a mean diameter of about 1100 Å.

In any of the embodiments discussed herein, the method may exclude a wash buffer, elution buffer, and/or mobile phase comprising NaCl, CaCl₂, or MgCl₂ (e.g., at a concentration of 250-500 mM).

In some embodiments of the methods, the heterodimeric protein is a bispecific antibody comprising a first polypeptide comprising a CH3 domain that is capable of binding to Protein A (“Fc”) and a second polypeptide comprising a CH3 domain that is not capable of binding to Protein A (“Fc*”). In some cases, the second polypeptide comprises a H435R/Y436F (by EU numbering system; H95R/Y96F by IMGT exon numbering system) substitution in its CH3 domain (a.k.a “Fc*” or “star substitution”). Thus, in some embodiments, the first homodimer is a monospecific antibody having two unsubstituted CH3 domains (i.e., FcFc); the second homodimer is a monospecific antibody having two H435R/Y436F substituted CH3 domains (i.e., Fc*Fc*); and the heterodimeric protein is a bispecific antibody having one unsubstituted CH3 domain and one H435R/Y436F substituted CH3 domain (i.e., Fc*Fc).

### EMBODIMENTS

In one or more aspects or embodiments thereof, this disclosure provides a method of purifying a heterodimeric protein from a mixture comprising the heterodimeric protein and impurities, the method comprising: (a) loading an affinity matrix containing a protein-binding ligand with a sample of the mixture, wherein the heterodimeric protein comprises first and second polypeptides with differing affinities for the protein-binding ligand, and wherein at least one impurity binds the protein-binding ligand with greater affinity than the heterodimeric protein and at least one impurity does not bind the protein-binding ligand or binds with lower affinity than the heterodimeric protein; (b) washing the mixture on the affinity matrix with a first mobile phase comprising a guanidinium salt at a concentration of from 50 mM to 300 mM; and (c) eluting the heterodimeric protein from the affinity matrix with a second mobile phase comprising an elution buffer.

In some embodiments, the heterodimeric protein is a bispecific antibody or an antigen-binding fragment thereof. In some embodiments, the impurities comprise homodimeric proteins.

In some embodiments of the methods disclosed herein, the guanidinium salt is a guanidinium cation paired with an anion selected from the group consisting of SO₄²⁻, HPO₄²⁻, acetate⁻, citrate⁻, Cl⁻, NO₃⁻, ClO₃⁻, I⁻, ClO₄⁻, and SCN⁻. In some embodiments, the guanidinium salt is guanidinium chloride. In some embodiments, the concentration of the guanidinium salt is 50 to 250 mM, about 50 mM, about 100 mM, about 150 mM, about 200 mM, or about 250 mM.

In some embodiments of the methods disclosed herein, the first mobile phase comprises a buffer. In some embodiments, the buffer is sodium acetate. In some embodiments, the buffer is at a concentration of from 100 mM to 300 mM. In some embodiments, the buffer is sodium acetate at a concentration of about 200 mM.

In some embodiments of the methods disclosed herein, the first mobile phase has a pH of 8-9, or about 8.5.

In some embodiments of the methods disclosed herein, the elution buffer contains an acid. In some embodiments, the acid is acetic acid, or wherein the elution buffer has a pH between 1 and 3. In some embodiments, the acetic acid is at a concentration of from 100 mM to 1 M, or wherein the acetic acid is at a concentration of about 500 mM.

In some embodiments of the methods disclosed herein the second mobile phase has a pH of from 3.5-5, 4-4.5, or 4.1-4.4.

In some embodiments of the methods disclosed herein, the protein-binding ligand is Protein A, and the first affinity matrix comprises a Protein A ligand affixed to a substrate. In some embodiments, the Protein A ligand is an engineered Protein A comprising a Z-domain tetramer, an engineered Protein A comprising a Y-domain tetramer, or an engineered Protein A that lacks D and E domains.

Some embodiments of the methods disclosed herein comprise a second affinity matrix. In some embodiments, a switch valves allows mobile phase flow switching between the first affinity matrix and the second affinity matrix. In some embodiments, the second affinity matrix comprises a second protein-binding ligand, and the affinity matrix comprises a Protein G ligand affixed to a substrate Protein G. In some embodiments, the substrate comprises any one or more of agarose, poly(styrene divinylbenzene), polymethacrylate, cellulose, controlled pore glass, and spherical silica.

In some embodiments of the methods disclosed herein, the first affinity matrix comprises a multiplicity of particles comprising a mean diameter of from 25 μm to 100 μm. In some embodiments, the second affinity matrix comprises a multiplicity of particles comprising a mean diameter of from 25 μm to 100 μm. In some embodiments, the particles comprise a mean diameter of from 40 μm to 60 μm. In some embodiments, the particles comprise a mean diameter of from 45 μm to 55 μm. In some embodiments, the particles comprise a mean diameter of about 50 μm. In some embodiments, the particles comprise pores having a mean diameter of about 1100 Å.

In some embodiments of the methods disclosed herein, the first polypeptide comprises a CH3 domain that is capable of binding to the protein-binding ligand and the second polypeptide comprises a CH3 domain that has reduced affinity for, or is not capable of binding to, the protein-binding ligand. In some embodiments, the first polypeptide comprises a CH3 domain that is capable of binding to Protein A and the second polypeptide comprises a CH3 domain that is not capable of binding to Protein A. In some embodiments, the second polypeptide comprises a HY to RF substitution in its CH3 domain.

In some embodiments of the methods disclosed herein, the first polypeptide and the second polypeptide comprise immunoglobulin constant domains of isotype IgG1. In some embodiments, the first polypeptide and the second polypeptide comprise immunoglobulin constant domains of isotype IgG4.

In one or more aspects or embodiments thereof, this disclosure provides a method of purifying a heterodimeric protein from a mixture comprising the heterodimeric protein and impurities, the method comprising: (a) loading an affinity matrix containing Protein A with a sample of the mixture, wherein the heterodimeric protein comprises first and second polypeptides with differing affinities for Protein A, and wherein at least one impurity binds the Protein A with greater affinity than the heterodimeric protein and at least one impurity does not bind the Protein A or binds the Protein A with lower affinity than the heterodimeric protein; (b) washing the mixture on the affinity matrix with a first mobile phase comprising guanidinium chloride and a buffer, wherein the guanidinium chloride is at a concentration of from 50 mM to 250 mM, and wherein the buffer is sodium acetate at a concentration of from 150 mM to 250 mM; and (c) eluting the heterodimeric protein from the affinity matrix with a second mobile phase comprising an elution buffer, wherein the elution buffer is acetic acid at a concentration of from 450 mM to 550 mM, and wherein the second mobile phase has a pH of 4.0 to 4.5.

In some embodiments of the methods disclosed herein, the heterodimeric protein is a bispecific antibody, wherein the bispecific antibody is an immunoglobulin molecule comprising four polypeptide chains, wherein the four polypeptide chains include two heavy chains and two light chains inter-connected by disulfide bonds, wherein each heavy chain comprises a heavy chain variable region and a heavy chain constant region comprising CH1, CH2 and CH3 domains, and wherein each light chain comprises a light chain variable region and a light chain constant region.

### EXAMPLES

### Example 1: Analysis of Homodimeric and Heterodimeric Proteins

An analysis of BsAb1 was performed. The sample containing a mixture of homodimeric and heterodimeric proteins was loaded onto a 1×0.2 mL MABSELECT SURE™ PCC ProA column. The ProA column was connected to a 1×0.1 mL POROS™ 20 μm Protein G column while a switch valve between the two columns allowed for skipping over one column and allowing flow through only the other column, or vice versa, as needed. Both columns were equilibrated in Mobile Phase 1 which contained 200 mM sodium acetate and 250 mM guanidinium chloride (GdmCl) at pH 8.5. Once loaded on the columns, samples were washed with Mobile Phase 1 to remove any unbound impurities. The different species in the samples were then eluted by modulating the pH of the mobile phase via addition of increasing amounts of a strip buffer containing 0.5 M acetic acid at pH 2.4. The ProG column was then taken offline by the switch valve to allow the elution to first come off the ProA column. The pH at which the FcFc* species elutes is molecule specific and, for example, can range from pH 4.0 to 4.5. FcFc* elutes at a higher pH due to its weaker binding to the ProA column, while the FcFc is stripped off the column at a much lower pH of 2.4. The ProA column was then equilibrated in Mobile Phase 1. The switch valve then brings back the ProG column back in line and the Fc*Fc* is eluted using the strip buffer at a pH of 2.4. After full elution, the system is then equilibrated back with Mobile Phase 1.

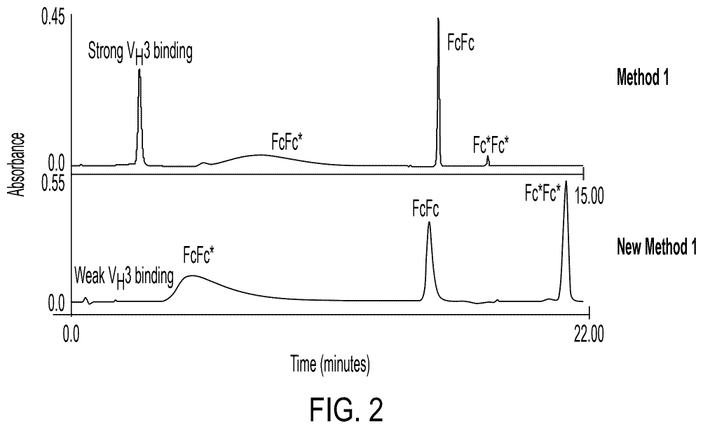

As shown in FIG. 2, the novel separation method, New Method 1, resulted in a chromatogram with minimal to no VH3 binding and clear resolution between and of the peaks of interest, i.e., the peaks corresponding to FcFc*, FcFc, and Fc*Fc*.

### Example 2: Comparison of Separation Methods

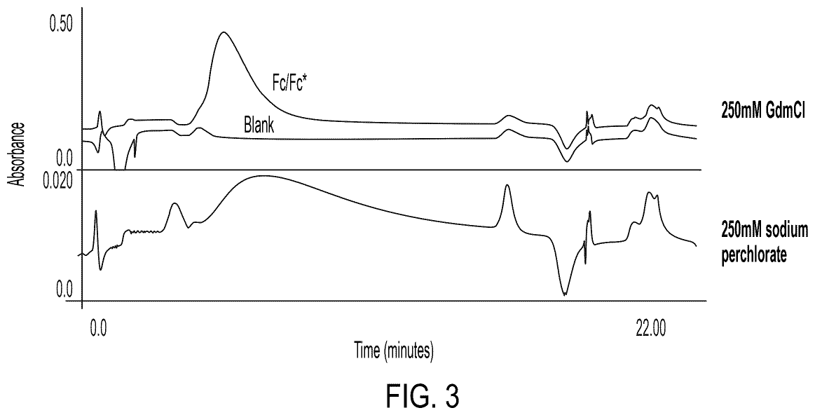

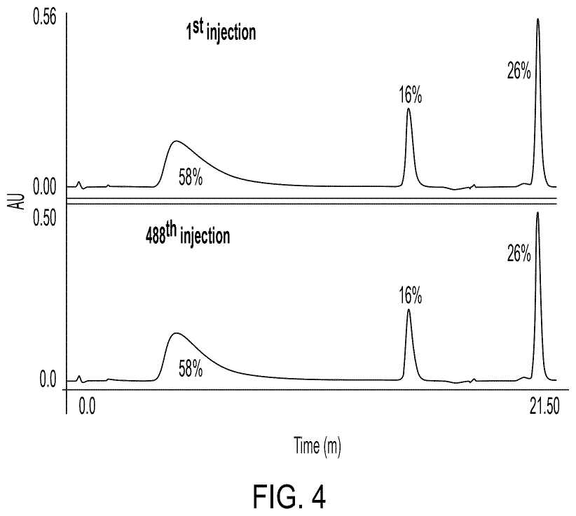

Table 1 summarizes and compares different examples of separation methods for isolating heterodimeric bispecific antibodies. Table 2 shows that a new method provides comparable quantification of titer standards as compared to other methods. GdmCl is an effective chaotropic substitute for CaCl₂) and lower concentrations of GdmCl can be used as compared to CaCl₂) without comprising resolution or separation of the peaks of interest, as shown in FIG. 2. For example, in Method 1, 500 mM CaCl₂) is used in the elution mobile phase but comparable separation results can be achieved with only 250 mM GdmCl. As shown in FIG. 3, 250 mM GdmCl in the elution mobile phase provides superior resolution than another salt, sodium perchlorate, at the same concentration. As shown in FIG. 4, the new methods maintain consistent and effective results over hundreds of injections demonstrating that separation columns need to be replaced less frequently.

TABLE 1
Various separation methods for analyzing bispecific antibodies.

| Method | Method 1 | Method 2 | New Method 1 |
|---|---|---|---|
| Number of Columns | 3 ProA | 2 ProA | 1 ProA |
| | 2 ProG | 2 ProG | 1 ProG |
| Method Time | ~10-15 min | ~30 min | ~22-35 min |
| Wash Mobile Phase | 20 mM Bis-Tris, | 20 mM Bis-Tris, | 200 mM sodium acetate, |
| | 500 mM NaCl, pH 6.4 | 500 mM NaCl, pH 64 | 250 mM GdmCl, pH 8.5 |
| Elution Mobile Phase | 200 mM sodium acetate, | 200 mM sodium acetate, | 200 mM sodium acetate, |
| | 500 mM CaCl₂, | 500 mM CaCl₂, | 250 mM GdmCl, |
| | 500 mM acetic acid | 500 mM acetic acid | 500 mM acetic acid |
| V_{H}3 Binding | High | Low | Low |

TABLE 2
Comparison of titer standards quantification between methods.

| | | |
|---|---|---|
| Bispecific | Method 1 or 2 | New Method 1 |

| Antibody | % Fc/Fc* | % Fc*Fc* | % Fc/Fc* | % Fc*Fc* |
|---|---|---|---|---|
| BsAb1 | 51 | 24 | 51 | 25 |
| BsAb2 | 46 | 34 | 42 | 38 |
| BsAb3 | 43 | 48 | 41 | 51 |
| BsAb4 | 38 | 52 | 35 | 53 |
| BsAb5 | 54 | 24 | 51 | 27 |

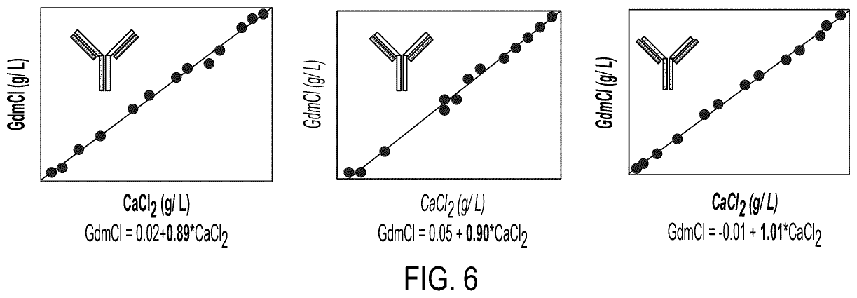

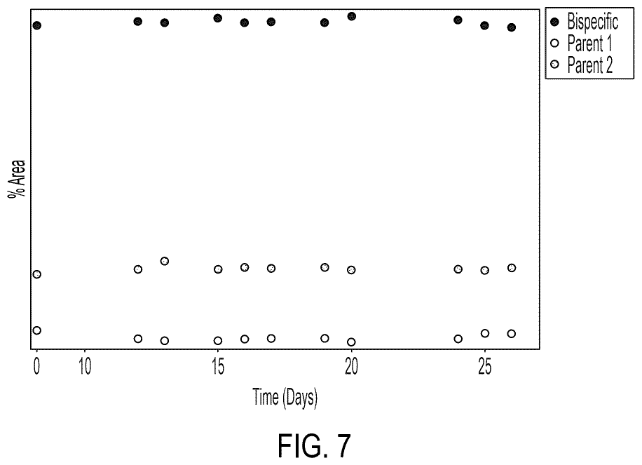

Table 3 summarizes the differences in chromatography instrument maintenance requirements between the methods using CaCl₂) or GdmCl as the chaotrope in the mobile phases. The use of CaCl₂) can lead to additional maintenance requirements. A method using GdmCl in place of CaCl₂) reduces maintenance requirements, even after prolonged use. The maintenance benefits afforded by the new method do not compromise sample data integrity (FIGS. 6 and 7). Comparable quantitation between the three antibody species is observed between methods using CaCl₂) in the mobile phase and the new method using GdmCl in the mobile phases, as shown in FIG. 6. Additionally, consistent analytical yields are observed in the new method even after over 500 injections over a period of 25 days, as shown in FIG. 7. A control sample was injected at regular intervals to monitor the performance of the instrument hardware during the collection of the data shown in FIG. 7. The control peak area percentage for each of the three species was used as a parameter to ensure that the instrument is performing consistently.

TABLE 3
Summary of Instrument Maintenance Requirements

| Method | Maintenance Requirement |
|---|---|
| Method 1 or 2 | Cleaning necessary approximately every 4 |
| | weeks to prevent instrument breakdowns |
| New Method 1 | No cleaning required over |
| | prolonged injection periods |

### Example 3: Screening Various Concentrations of GdmCl in the Analysis of a Bispecific Antibody

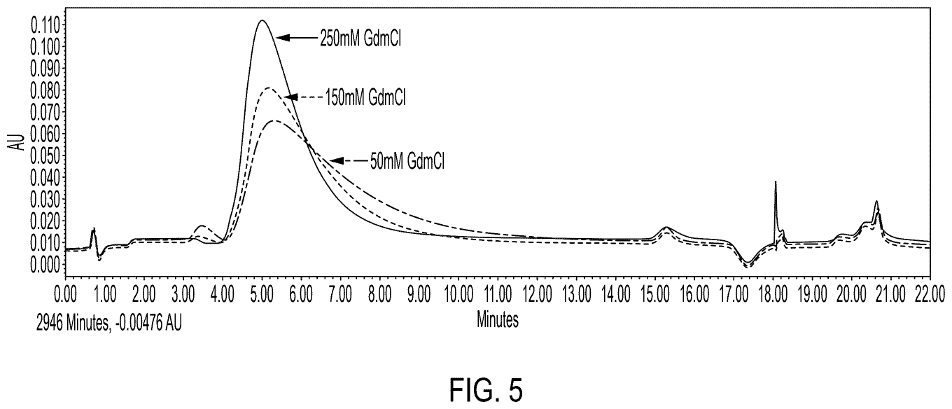

Analytical protocols were tested with various concentrations of GdmCl to compare chromatogram peak shape and peak area. Here, the wash phase used contained 20 mM Bis-Tris and 500 mM NaCl at pH 6.4. The elution phase contained 200 mM sodium acetate, 250 mM GdmCl, and 500 mM acetic acid. In the elution phase, concentrations of 50 mM, 100 mM, 150 mM, and 200 mM GdmCl were compared. The injection volume (10 μL) of sample loaded on the column between the different GdmCl concentrations trials was the same. The results of this assessment are shown in FIG. 5.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method of analyzing a heterodimeric protein in a mixture comprising the heterodimeric protein and impurities, the method comprising:
(a) loading an affinity matrix containing a protein-binding ligand with a sample of the mixture, wherein the heterodimeric protein comprises first and second polypeptides with differing affinities for the protein-binding ligand, and wherein at least one impurity binds the protein-binding ligand with greater affinity than the heterodimeric protein and at least one impurity does not bind the protein-binding ligand or binds with lower affinity than the heterodimeric protein;
(b) washing the mixture on the affinity matrix with a first mobile phase comprising a guanidinium salt at a concentration of from 50 mM to 300 mM;
(c) eluting the heterodimeric protein in an eluate from the affinity matrix with a second mobile phase comprising an elution buffer; and
(d) quantitating the heterodimeric protein in the eluate.

2. The method of claim 1, wherein the heterodimeric protein is a bispecific antibody or an antigen-binding fragment thereof.

3. The method of claim 1, wherein the impurities comprise homodimeric proteins.

4. The method of claim 1, wherein the guanidinium salt is a guanidinium cation paired with an anion selected from the group consisting of SO₄²⁻, HPO₄²⁻, acetate⁻, citrate⁻, Cl⁻, NO₃⁻, ClO₃⁻, I⁻, ClO₄⁻, and SCN⁻.

5. The method of claim 1, wherein the guanidinium salt is guanidinium chloride.

6. The method of claim 1, wherein the concentration of the guanidinium salt is 50 to 250 mM, about 50 mM, about 100 mM, about 150 mM, about 200 mM, or about 250 mM.

7. The method of claim 1, wherein the first mobile phase further comprises a buffer.

8. The method of claim 7, wherein the buffer is sodium acetate.

9. The method of claim 7, wherein the buffer is at a concentration of from 100 mM to 300 mM.

10. The method of claim 7, wherein the buffer is sodium acetate at a concentration of about 200 mM.

11. The method of claim 7, wherein the first mobile phase has a pH of 8-9, or about 8.5.

12. The method of claim 1, wherein the elution buffer contains an acid.

13. The method of claim 12, wherein the acid is acetic acid, or wherein the elution buffer has a pH between 1 and 3.

14. The method of claim 12, wherein the acetic acid is at a concentration of from 100 mM to 1 M, or wherein the acetic acid is at a concentration of about 500 mM.

15. The method of claim 1, wherein the second mobile phase has a pH of from 3.5-5, 4-4.5, or 4.1-4.4.

16. The method of claim 1, wherein the protein-binding ligand is Protein A, and the first affinity matrix comprises a Protein A ligand affixed to a substrate.

17. (canceled)

18. The method of claim 1, further comprising a second affinity matrix.

19. (canceled)

20. The method of claim 18, wherein the second affinity matrix comprises a second protein-binding ligand, and the affinity matrix comprises a Protein G ligand affixed to a substrate Protein G.

21.-32. (canceled)

33. A method of analyzing a heterodimeric protein in a mixture comprising the heterodimeric protein and impurities, the method comprising:
(a) loading an affinity matrix containing Protein A with a sample of the mixture, wherein the heterodimeric protein comprises first and second polypeptides with differing affinities for Protein A, and wherein at least one impurity binds the Protein A with greater affinity than the heterodimeric protein and at least one impurity does not bind the Protein A or binds the Protein A with lower affinity than the heterodimeric protein;
(b) washing the mixture on the affinity matrix with a first mobile phase comprising guanidinium chloride and a buffer, wherein the guanidinium chloride is at a concentration of from 50 mM to 250 mM, and wherein the buffer is sodium acetate at a concentration of from 150 mM to 250 mM; and
(c) eluting the heterodimeric protein from the affinity matrix in an eluate with a second mobile phase comprising an elution buffer, wherein the elution buffer is acetic acid at a concentration of from 450 mM to 550 mM, and wherein the second mobile phase has a pH of 4.0 to 4.5; and
(d) quantitating the heterodimeric protein in the eluate.

34. (canceled)

35. A method of analyzing a heterodimeric protein and two types of homodimeric proteins in a mixture comprising the heterodimeric protein and the two types of homodimeric proteins, the method comprising:
(a) loading a first affinity matrix containing a first protein-binding ligand with a sample of the mixture, wherein the heterodimeric protein comprises first and second polypeptides with differing affinities for the first protein-binding ligand, wherein a first type of homodimeric protein binds the protein-binding ligand with greater affinity than the heterodimeric protein and a second type of homodimeric protein does not bind the protein-binding ligand or binds with lower affinity than the heterodimeric protein, and wherein the first protein-binding ligand is Protein A;
(b) loading a second affinity matrix containing a second protein-binding ligand with a sample of the mixture, wherein the second type of homodimeric protein binds the second protein-binding ligand with greater affinity than the heterodimeric protein, and wherein the second protein-binding ligand is Protein G;
(c) washing the mixture on the affinity matrix with a first mobile phase comprising a guanidinium salt at a concentration of from 50 mM to 300 mM and a buffer, wherein the buffer is sodium acetate at a concentration of from 100 to 300 mM sodium acetate;
(d) eluting the heterodimeric protein and the first type of homodimeric protein from the first affinity matrix with a second mobile phase comprising an elution buffer, wherein the elution buffer comprises acetic acid at a concentration of from 300 mM to 700 mM mixed with the first mobile phase in increasing volumes to decrease pH of the first mobile phase over time;
(e) eluting the second type of homodimeric protein from the second affinity matrix; and
(f) quantitating the first type of homodimeric protein, the heterodimeric protein, and the second type of homodimeric protein.

36.-41. (canceled)
